(19) United States
(12) Patent Application Publication
(10) Pub. No.: US 2010/0172472 A1
(43) Pub. Date: Jul. 8, 2010

(54) COLLECTING IMAGES FOR IMAGE STITCHING WITH ROTATING A RADIATION DETECTOR

(75) Inventor: Jean-Pierre Franciscus Alexander Maria Ermes, Eindhoven (NL)

Correspondence Address: PHILIPS INTELLECTUAL PROPERTY & STANDARDS, P.O. BOX 3001, BRIARCLIFF MANOR, NY, 10510, US

(73) Assignee: KONINKLIJKE PHILIPS ELECTRONICS N.V., EINDHOVEN (NL)

(21) Appl. No.: 12/377,009

(22) PCT Filed: Aug. 9, 2007
(86) PCT No.: PCT/IB2007/053163

§ 371 Date: Feb. 10, 2009

(30) Foreign Application Priority Data
Aug. 14, 2006 (EP) 06118870.2

(51) Int. Cl. G01N 23/04; G06K 9/36; G01J 1/42; A61B 6/00

(52) U.S. Cl. 378/62; 382/284; 250/395; 250/336.1

(57) ABSTRACT

It is described a method for extending the imaged area of an imaging apparatus (100) by stitching several images (211, 212) together. The method comprises acquiring two images (211, 212) showing different parts of one and the same object (107, 207). Thereby, during both image acquisitions the spatial relationship between a radiation source (104, 204) and the object (107, 207) is maintained constant. Further, in between the two image acquisitions a radiation detector (105, 205) is rotated around the radiation source (104, 204). The method minimizes the stitching deformations by using a new arrangement of the image-acquisition geometries. A customized stitching algorithm can correct for small remaining distortions and yield a perfect perspective projection of the who Ie overview.

The present invention relates to the field of digital image processing, in particular the present invention relates to digital image processing for medical purposes, wherein an enlarged image is generated by means of a stitching procedure performed with two or more images representing different field of views of one and the same object.

Specifically, the present invention relates to a method for collecting images of an object of interest for the purpose of image stitching in order to provide for an enlarged image field of view.

Further, the present invention relates to a data processing device and to a medical system for collecting images of an object of interest for the purpose of image stitching in order to provide for an enlarged image field of view.

Furthermore, the present invention relates to a computer-readable medium and to a program element having instructions for executing the above-mentioned method for collecting images of an object of interest for the purpose of image stitching.

In many X-ray imaging systems, an X-ray source projects an area beam which is collimated to pass through an object of interest being imaged, such as a patient. The X-ray beam, after being attenuated by the object, impinges upon an array of radiation detectors. The intensity of the radiation beam received at the detector array is dependent upon the attenuation of the X-ray beam by the object. In a digital detector, each detector element or pixel of the array produces a separate electrical signal that is a measurement of the beam attenuation at that location of the detector. The attenuation measurements from all the detector pixels are acquired separately to produce a transmission profile representing a two-dimensional image.

In X-ray imaging there are applications wherein an X-ray image is generated having a larger field of view than the field of view defined by the geometry of the X-ray imaging system, such as the positions of the radiation source, the object of interest and the radiation detector and in particular by the two dimensional dimensions of the radiation detector. In order to enlarge the field of view of an X-ray imaging system there are known image stitching methods. Image stitching, or the creation of a composite image, is usually accomplished obtaining different images of one and the same object and to paste these images together. Thereby, between two images there is usually used an overlap in order to allow for a correct relative positioning of the two images.

U.S. Pat. No. 6,898,269 B2 discloses a method for producing an image in an X-ray imaging system. The X-ray imaging system includes an X-ray source which projects an X-ray beam collimated by a collimation assembly to pass through an object of interest and impinge onto an X-ray receptor to produce the image. The method includes rotating the collimation assembly about a focal point while the X-ray source is substantially kept in a fixed position. The method further includes adjusting the position of the X-ray receptor during rotation of the collimation assembly to receive the x-ray beam.

EP 1 484 016 A1 discloses a control of an X-ray system in order to obtain a view of an area of a patient that is larger than a field of view of an X-ray detector. Individual images are obtained of portions of the area of the patient that, when combined, can be used to get an enlarged view of the area of the subject. Positions of individual images are determined. These positions are preferably calculated in order to avoid placing structures that tend to move or that are dose sensitive in an area of overlap of the individual images. Also, the positions are preferably calculated to reduce overall exposure to a subject, especially by reducing unnecessary double exposure. Further, positions of the X-ray detector necessary to obtain the individual images are calculated in order to hold a relative location between the patient and the X-ray source constant while the images are being collected. The position of the X-ray detector is controlled with a control signal to collect the images based on the calculated positions.

US 2004/0101103 A1 discloses a method for collecting X-ray images for image pasting using a device having an X-ray source and a flat-panel X-ray detector using a field of view. The steps in the method include obtaining a first image of a subject of interest at a first position using X-rays transmitted through the subject of interest detected by the flat panel X-ray detector; moving the detector a distance no more than a length of a field of view of the detector in a direction of the movement; obtaining a second image of the subject of interest at a second position using X-rays transmitted through the subject of interest detected by the flat panel X-ray detector; and joining the first and second images at a line of overlap to form a pasted image having an image field of view larger than the field of view of the detector.

U.S. Pat. No. 5,712,890 discloses a digital X-ray mammography device, which is capable of imaging a full breast. A movable aperture coupled with a movable X-ray image detector permits X-ray image data to be obtained with respect to partially overlapping X-ray beam paths from an X-ray source passing through a human breast. A digital computer programmed with a stitching algorithm produces a composite image of the breast from the image data obtained with respect to each path.

A problem with all these current known image-stitching methods and the corresponding devices is that they usually do not give high quality images making the stitched image much less accurate than the original images.

There may be a need for an improved image stitching providing for high quality stitched images.

This need may be met by the subject matter according to the independent claims. Advantageous embodiments of the present invention are described by the dependent claims.

According to a first aspect of the invention there is provided a method for collecting images of an object of interest for the purpose of image stitching in order to provide for an enlarged image field of view. The provided method comprises (a) acquiring a first image of the object using first radiation being emitted from a radiation source, being transmitted through the object and being detected by a radiation detector, whereby the object is positioned relative to the radiation source in a first spatial position, (b) rotating the radiation detector around the radiation source, and (c) acquiring a second image of the object using second radiation being emitted from the radiation source, being transmitted through the object and being detected by the radiation detector, whereby the object is positioned relative to the radiation source in a second spatial position, which is the same as the first spatial position.

This aspect of the invention is based on the idea that image stitching deformations may be minimized by using the knowledge of the X-ray acquisition geometry. This means that the spatial positions of the radiation source, the object and the radiation detector relative to each other are is known exactly during each image acquisition.

According to the provided method for both image acquisitions the radiation source has the same relative position with respect to the object. In between the two image acquisitions the radiation detector is rotated around the radiation source. This means that during the acquisition of the first image the radiation detector is positioned relative to the radiation source in a first spatial position, whereas during the acquisition of the second image the radiation detector is positioned relative to the radiation source in a second spatial position.

The provided method allows for collecting images, which can be stitched together in order to form a composed image, which is much larger than the dimensions of the radiation detector. Preferably, the detector is a detector array having a length and a width, which allows for a field of view, which already covers a significant portion of the object of interest.

However, it has to be pointed out that the described method may also be carried out with a line sensor, wherein the length of the line is shorter than at least one dimension of the object. A two dimensional image may be obtained by repeating the described method for a variety of different lateral displacements of the object with respect to the radiation source.

According to an embodiment of the present invention the step of rotating the radiation detector is carried out in a circular manner. This has the advantage that a rather simple mechanical movement is sufficient in order to carry out the described method. Preferably, the mechanical movement is carried out with a rotatable gantry, wherein the radiation detector is fixed to the gantry.

According to a further embodiment of the invention the step of rotating the radiation detector comprises maintaining the spatial position of the object relative to the radiation source. This may provide the advantage that, in case also the radiation source is moved in between acquiring the first image and acquiring the second image, the radiation source movement can be simultaneously compensated by a mutual movement of the object in order to compensate the movement of the radiation source. Therefore, no sequential movements have to be accomplished such that the data acquisition of the second image can be started immediately after the radiation source has been reached its final position.

According to a further embodiment of the invention the step of rotating the radiation detector comprises rotating both (a) the radiation detector and the radiation source around a rotational axis, and (b) translating the object relative to the rotational axis such that the second spatial position is the same as the first spatial position. This has the advantage that the described method may be carried out with standard X-ray systems such as a C-arm or a computed tomography (CT) system, wherein the radiation detector and the radiation source are rotatable around a common virtual rotational axis. In this respect virtual means that there is no shaft arranged physically in the rotational axis but there is a rotation assembly being formed around the rotational axis.

The translation of the object relative to the rotational axis may be carried out by means of a positioning device which is adapted to move a table whereon the object, e.g. a patient, is positioned. However, the translation of the object relative to the rotational axis may also be carried out by moving the X-ray system and/or by moving both the object and the X-ray system. Anyway, a sole rotation of the radiation detector around the radiation source has to be imitated or mimicked.

The rotation of the radiation source has the further advantage that the radiating being emitted may be always directed straight onto the radiation detector even if the angle of beam spread is limited. In other words, most of the radiation being emitted from the radiation source can be employed both for acquiring the first image and for acquiring the second image.

According to a further embodiment of the invention the described method further comprises joining the first image and the second image at a region of overlap to form a stitched image having an image field of view larger than the field of view of the first image or second image individually.

The described rotation of the radiation detector may provide the advantage that depth differences within the object will not result in artifacts of the stitched image. Therefore, the overlap, which is necessary in order to reliably stitch the two images together, can be minimized such that the field of view of the resultant stitched respectively combined image is almost doubled compared to the field of view of the first respectively the second image.

When the geometry of the X-ray acquisition is known, an image-stitching algorithm can mimic the perfect perspective projection allowing for an image reconstruction with a quality similar to the image quality of an obtained single.

In this respect is has to be pointed out that the described method also allows for joining three or even more images. This has the advantage that the resultant field of view may be enlarged even more significantly. In case three or even more images are combined in a spatial sequence, preferably the distance between the radiation source and the radiation detector is large enough such that scaling differences and/or optical distortions within the combined image are kept with acceptable limits.

According to a further embodiment of the invention the step of joining the first image and the second image comprises determining the relative position between the first image and the second image by using a common geometry being identifiable within both the first image and the second image. This may provide the advantage that joining or stitching the two images may be carried out automatically by means of known image processing algorithms.

According to a further embodiment of the invention the described method further comprises resampling data representing the first image and/or resampling data representing the second image in order to simulate a planar common virtual detector plane for acquiring a first resampled image and for acquiring a second resampled image.

The described stitching method for joining different display windows may provide the advantage that scaling differences within the stitched respectively the composed image are reduced significantly. Such scaling differences are typically caused by non-uniform distances between the radiation source and the object and between the object and the radiation detector, respectively.

In this respect resampling means that each pixel in the resampled image is reconstructed by taking into account the known geometric arrangement of radiation source, object and radiation detector during the entire image acquisition. Thereby, for each pixel of the resampled image the intersection of (a) the corresponding radiation ray originating from the radiation source and impinging onto this pixel with (b) the original source image being represented by the radiation detector is calculated. The corresponding value (e.g. a grey scale value) of this pixel can be found by an interpolation of the surrounding source pixels.

Preferably, this virtual detector plane is oriented parallel to the object. This has the advantage that stitching the resampled images will result in a perfect perspective projection of the extended field of view of the stitched image.

According to a further embodiment of the invention the first radiation and/or the second radiation is X-radiation. This has the advantage that the described method may be employed for X-ray imaging, wherein portions of the object are X-ray imaged, which portions are larger than the field of view being limited in particular by the detector size. Therefore, the described method provides for a simple and for an effective enlargement of the field of view of many X-ray imaging systems.

The described method may be used in particular for medical X-ray imaging of body parts that extent the size of the available radiation detector. Preferably, the described method may be used for X-ray imaging of the pelvis or imaging of both shoulders. However, the acquisitions with a rotated X-ray detector can also be done in the longitudinal direction of the patient such that one can image at least parts of the spine or the legs.

According to a further aspect of the invention there is provided a data processing device for collecting images of an object of interest for the purpose of image stitching in order to provide for an enlarged image field of view. The data processing device comprises (a) a data processor, which is adapted for performing exemplary embodiments of the above-described method, and (b) a memory for storing image data representing the first and/or the second image.

According to a further aspect of the invention there is provided a medical system, in particular a C-arm system, for collecting images of an object of interest for the purpose of image stitching in order to provide for an enlarged image field of view. The medical system comprises the above described a data processing device.

It has to be pointed out that in addition to the radiation source and the radiation detector the medical system may comprise an X-ray intensifier. In this respect it is clear that all constraints mentioned above regarding the positioning of the radiation detector in this case have to be applied to the positioning of the X-ray intensifier.

According to a further aspect of the invention there is provided a computer-readable medium on which there is stored a computer program for collecting images of an object of interest for the purpose of image stitching in order to provide for an enlarged image field of view. The computer program, when being executed by a data processor, is adapted for performing exemplary embodiments of the above-described method.

According to a further aspect of the invention there is provided a program element for collecting images of an object of interest for the purpose of image stitching in order to provide for an enlarged image field of view. The program element, when being executed by a data processor, is adapted for performing exemplary embodiments of the above-described method.

The computer program element may be implemented as computer readable instruction code in any suitable programming language, such as, for example, JAVA, C++, and may be stored on a computer-readable medium (removable disk, volatile or non-volatile memory, embedded memory/processor, etc.), the instruction code operable to program a computer of other such programmable device to carry out the intended functions. The computer program may be available from a network, such as the WorldWideWeb, from which it may be downloaded.

It has to be noted that embodiments of the invention have been described with reference to different subject matters. In particular, some embodiments have been described with reference to method type claims whereas other embodiments have been described with reference to apparatus type claims. However, a person skilled in the art will gather from the above and the following description that, unless other notified, in addition to any combination of features belonging to one type of subject matter also any combination between features relating to different subject matters, in particular between features of the method type claims and features of the apparatus type claims is considered to be disclosed with this application.

The aspects defined above and further aspects of the present invention are apparent from the examples of embodiment to be described hereinafter and are explained with reference to the examples of embodiment. The invention will be described in more detail hereinafter with reference to examples of embodiment but to which the invention is not limited.

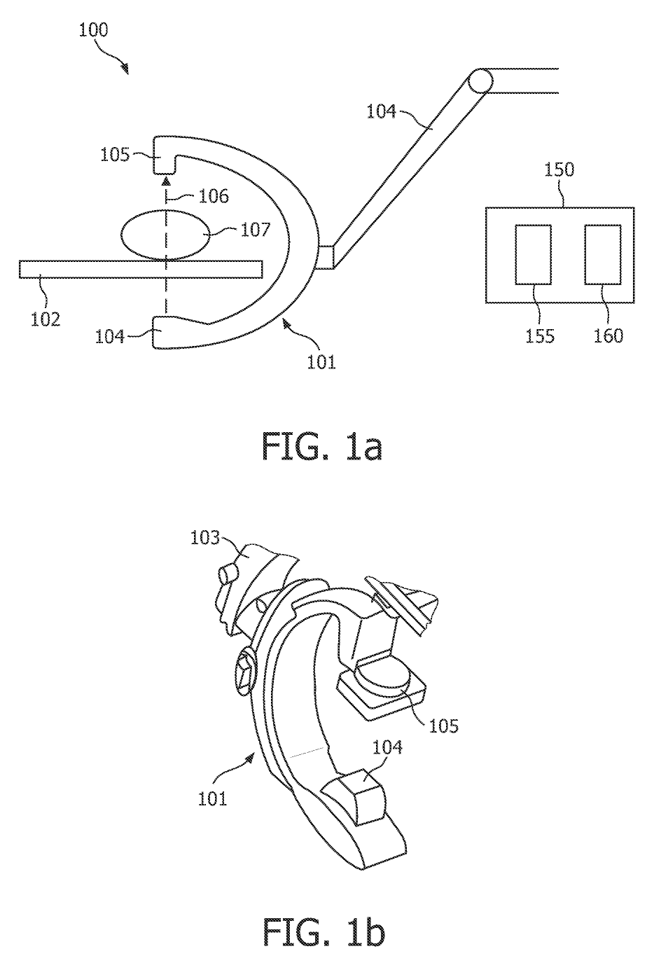

FIG. 1a shows a schematic side view of a medical C-arm system.

FIG. 1b shows a perspective view of the X-ray swing arm shown in FIG. 1a.

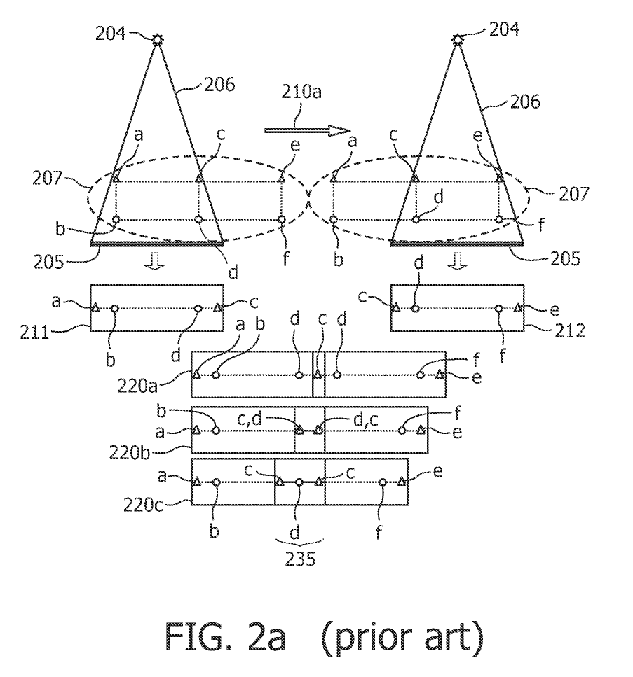

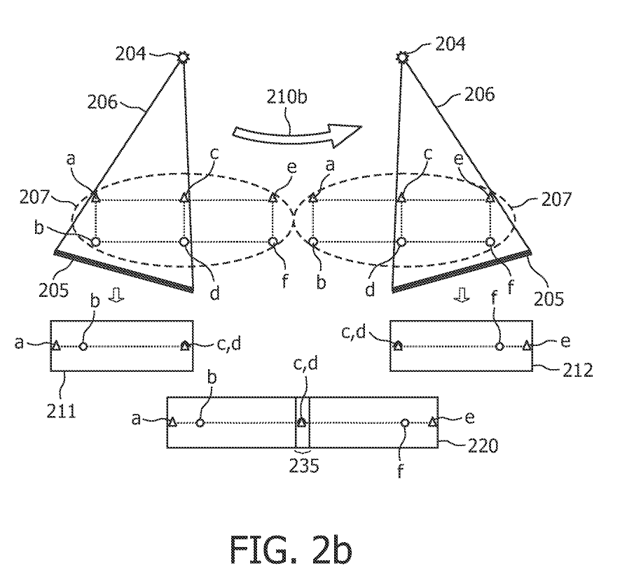

FIG. 2a illustrates a known stitching procedure of two images obtained by a translation of an object of interest with respect to a imaging system comprising a radiation source and a radiation detector.

FIG. 2b illustrates a stitching procedure according to an embodiment of the invention, wherein two images are obtained by means of a rotation of the radiation detector around the radiation source.

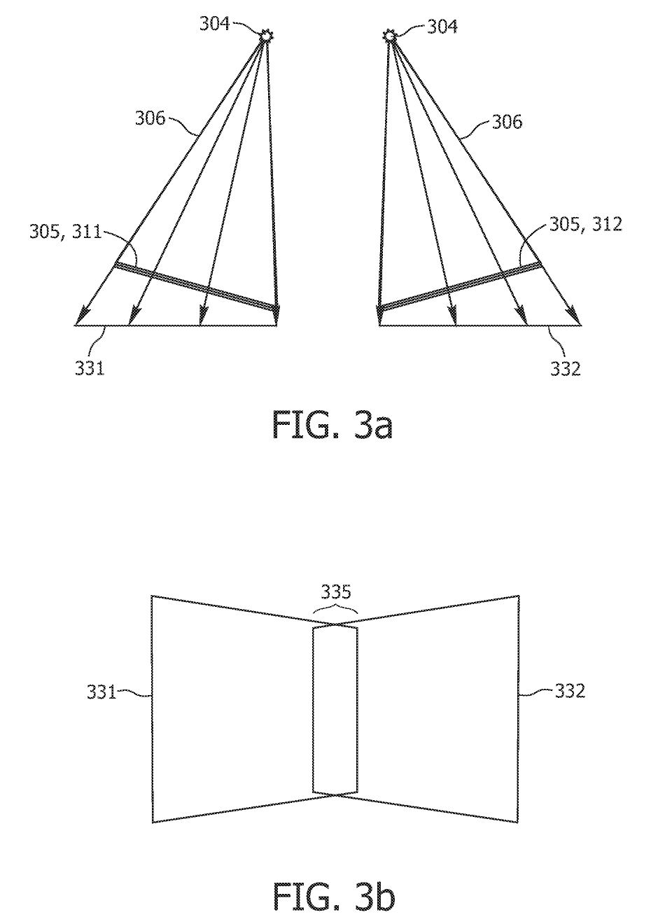

FIG. 3a illustrates the procedure of resampling an image by means of a projection towards a slanted plane.

FIG. 3b illustrates a stitching of two resampled images.

The illustration in the drawing is schematically. It is noted that in different figures, similar or identical elements are provided with the same reference signs or with reference signs, which are different from the corresponding reference signs only within the first digit.

Referring to FIGS. 1a and 1b of the drawing, a medical X-ray imaging system 100 according to an embodiment of the invention comprises a swing arm scanning system (C-arm) 101 supported proximal a patient table 102 by a robotic arm 103. Housed within the swing arm 101, there is provided an X-ray tube 104 and an X-ray detector 105, the X-ray detector 105 being arranged and configured to receive X-rays 106, which have passed through a patient 107. Further, the X-ray detector 105 is adapted to generate an electrical signal representative of the intensity distribution thereof. By moving the swing arm 101, the X-ray tube 104 and the detector 105 can be placed at any desired location and orientation relative to the patient 107.

The C-arm system 100 further comprises a control unit 155 and a data processing device 160, which are both accomodetaed within a workstation or a personal computer 150. The control unit 155 is adapted to control the operation of the C-arm system 100. The data processing device 160 is adapted for collecting images of the object 107 for the purpose of image stitching in order to provide for an enlarged image field of view of the patient 107.

In the following there is described a stitching method for pasting two images with each other such that a combined image having an enlarged field of view is generated. The stitching method representing an embodiment of the invention is described with reference to FIG. 1b. In order to facilitate the understanding of the described stitching method, there is first described a known stitching method with reference to FIG. 1a.

As can be seen from FIG. 1a, a radiation source 204 emits a radiation beam 206 penetrating a left part of an object of interest 207, e.g. a patient. The spatial intensity distribution of the transmitted radiation beam 206 is detected by means of a radiation detector 205, which is a two dimensional detector array comprising a plurality of detector elements (detector pixels). A two-dimensional first image 211 of the left part of the object 207 is acquired. Within this left part there are depicted four exemplary voxels a, b, c and d, which are spatially arranged within the three-dimensional object 207. Thereby, the voxels a and c are arranged on an upper line traversing the object 207 in a horizontal direction. The voxels b and d are arranged on a lower line also traversing the object 207 in a horizontal direction. Further, the voxels a and b are arranged on a left line traversing the object 207 in a vertical direction. The voxels c and d are arranged on a middle line traversing the object 207 also in a vertical direction.

Due to the angle of beam spread of the radiation beam 206 the voxels a and b appear on the image 211 with a lateral offset with respect to each other. The same holds for the voxels c and d. Of course, the magnitude of the lateral offset depends on the vertical distance between the voxels a and b and c and d, respectively. Furthermore, the offset depends on the position of the voxels with respect to a not depicted optical axis of the radiation beam 206, which optical axis extends between the radiation source 204 and the center of the detector 205.

After the first image 211 has been acquired, the object 207 is linearly shifted with respect to both the detector 205 and the radiation source 204. This is indicated by the arrow 210a indicating this translatory shift.

As can be seen from the right part of FIG. 2a, in the shifted position the right part of the object 207 is illuminated by means of the radiation beam 206. The right part of the object comprises the voxels c, d and further exemplary voxels e and f. Thereby, the voxels c and e are arranged on an upper line traversing the object 207 in a horizontal direction. The voxels d and f are arranged on a lower line also traversing the object 207 in a horizontal direction. Further, as has already been mentioned the voxels c and d are arranged on the middle line traversing the object 207 in a vertical direction and the voxels e and f are arranged on a right line traversing the object 207 also in a vertical direction.

Due to the angle of beam spread of the radiation beam 206 mentioned already above, the voxels c and d appear on the image 212 with a lateral offset with respect to each other. The same holds for the voxels e and f. Again, the magnitude of the lateral offset depends on the vertical distances between each two voxels and on the position of the corresponding voxels with respect to the not depicted optical axis.

After the images 211 and 212 have been obtained, there are in particular three different prominent ways in order to stitch or to combine these images. Thereby, within an offset region 235 different voxels are superimposed. If one superimposes the voxel c of both images 211 and 212 one obtains the composed image 220a. Therein, the voxel d is included twice. This means that the image quality of the combined image 220a in the offset region is very poor.

If one superimposes the voxel c of the image 211 with the voxel d of the image 212 and vice versa one obtains the stitched image 220b. Also here the image quality is very poor in particular in the offset region 235 because both voxels c and d each appear on two positions. The same holds if one superimposes the voxel d of both images 211 and 212 in order to obtain the composed image 220c. Therein, the voxel c is included twice such that also the composed image 220c exhibits a poor image quality.

By contrast to the translative movement of the object 207, according to the embodiment of the invention described here with reference to FIG. 2b, the first image 211 and the second image 212 are acquired whereby during each image acquisition the object 207 is positioned relative to the radiation source 204 in the same spatial position. After the left part of the object 207 including the voxels a, b, c and d has been imaged, the radiation detector 205 is rotated around the radiation source 204 in a circular manner. This rotation is indicated by the arrow 210b.

Since within both images 211 and 212 the two voxels c and d are superimposed, a stitching of the two images 211 and 212, wherein within the overlap 235 these voxels are also superimposed, leads to a combined image 220. As can be gathered from the defined overlap region 235, the quality of the combined image 220 is much better than the quality of the stitched images 220a, 220b and 220c.

It has to be mentioned that of course also the radiation source 204 and/or a non-depicted collimator assembly might also be rotated preferably following the rotation of the radiation detector 205. However, the spatial position of a focal point of the radiation source, i.e. the point representing the origin of all radiation rays 206, has to be kept in a fixed position with respect to the object 207.

The described rotational movement of the radiation detector 207 is preferably realized by means of a C-arm system. Thereby, both the radiation detector 207 and the radiation source 204 are mounted at a C-arm, which is rotatable around a rotational axis. In order to compensate for the movement of the radiation source 204, the object 207, e.g. a patient, has to be moved in a translative manner such that the relative spatial positioning between the radiation source 204 and the object 207 is maintained.

The translation of the object 207 relative to the rotational axis may be carried out by means of a positioning device which is adapted to move a table whereon the object 207 is positioned. However, the translation of the object 207 relative to the rotational axis may also be carried out by moving the X-ray system and/or by moving both the object 207 and the X-ray system. Anyway, a sole rotation of the radiation detector 205 around the radiation source 204 has to be imitated.

It has to be mentioned that when both images 211 and 212 acquired by means of the rotated radiation detector 205 are stitched together, there will remain a small scaling difference in the overview image 220 due to the non-uniform distances between the radiation source and the object and between the object and the radiation detector, respectively.

This has the effect that given a typical geometry of source-image distance of 150 cm, a 30 cm detector size and an overlap 235 between both images 211 and 212 of 5 cm, it can be derived that the scaling difference between the centre of the overview image and its borders is about 1.5%. This means that within the stitched image 220 the length of a rod being oriented horizontally parallel to the patient and having a real length of 10 cm varies about 1.5 mm depending on its position within the composed image 220.

By contrast to a stitching of translated images (see FIG. 2a), with the same acquisition geometry and a 10 cm rod placed in a vertical orientation perpendicular to the patient, within the overlap region 235 double contour artifacts of about 10 mm are generated. Therefore, the typical errors, which are produced when rotated images are stitched, are in an order of magnitude smaller than the errors, which are produced when rotated images are stitched together.

However, the above described residual scaling difference with the stitched image 220 can even be compensated for by resampling the images 211 and 212 towards the patient plane. In the following this resampling will be described with reference to FIGS. 3a and 3b.

As can be seen from FIG. 3a depicting a preferred embodiment for a resampling procedure, the resampling is carried out by projecting the images 311 and 312 acquired by means of the radiation detector 305 towards slanted planes comprising resampled images 331 and 332, respectively. Thereby, a planar common virtual detector plane is simulated for acquiring the first image 311 and for acquiring the second image 312.

In this respect resampling means that each pixel in the resampled image is reconstructed by taking into account the known geometric arrangement of radiation source, object and radiation detector during the image acquisition. Thereby, for each pixel of the resampled image 331, 332 the intersection of (a) the corresponding radiation ray 306 originating from the radiation source 304 and impinging onto this pixel with (b) the original source image 311, 312 being represented by the radiation detector 305 is calculated. The corresponding value (e.g. a grey scale value) of this pixel can be found by an interpolation of the surrounding source pixels.

As can be seen from FIG. 3a, this virtual detector plane is oriented parallel to the object (not depicted in FIG. 3a). This has the advantage that stitching the resampled images 331, 332 will result in a perfect perspective projection of the extended field of view of the stitched image.

FIG. 3b shows a schematic representation of two resampled images 331 and 332. The corresponding source images have been acquired by means of a detector array having the shape of a rectangle. Due to the resampling onto a slanted plane the resampled images 331 and 332 each have the shape of a trapeze. The resampled images 331 and 332 are stitched together with an overlap 335.

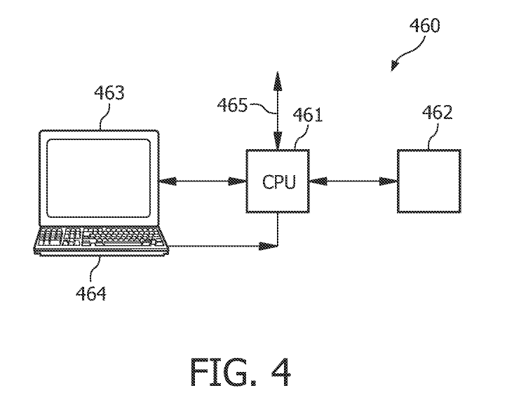

FIG. 4 depicts an exemplary embodiment of a data processing device 460 according to the present invention for executing an exemplary embodiment of a method in accordance with the present invention. The data processing device 460 comprises a central processing unit (CPU) or image processor 461. The image processor 461 is connected to a memory 462 for temporally storing acquired or processed datasets. Via a bus system 465 the image processor 461 is connected to a plurality of input/output network or diagnosis devices, such as a CT scanner or preferably a C-arm being used for two-dimensional X-ray imaging. Furthermore, the image processor 461 is connected to a display device 463, for example a computer monitor, for displaying stitched images. An operator or user may interact with the image processor 461 by means of a keyboard 464 and/or by means of any other output devices, which are not depicted in FIG. 4.

It should be noted that the term “comprising” does not exclude other elements or steps and the “a” or “an” does not exclude a plurality. Also elements described in association with different embodiments may be combined. It should also be noted that reference signs in the claims should not be construed as limiting the scope of the claims.

In order to recapitulate the above described embodiments of the present invention one can state:

It is described a method for extending the imaged area of an imaging apparatus 100 by stitching several images 211, 212 together. The method comprises acquiring two images 211, 212 showing different parts of one and the same object 107, 207. Thereby, during both image acquisitions the spatial relationship between a radiation source 104, 204 and the object 107, 207 is maintained constant. Further, in between the two image acquisitions a radiation detector 105, 205 is rotated around the radiation source 104, 204. The method minimizes the stitching deformations by using a new arrangement of the image-acquisition geometries. A customized stitching algorithm can correct for small remaining distortions and yield a perfect perspective projection of the whole overview.

### LIST OF REFERENCE SIGNS

100 medical X-ray imaging system/C-arm system

101 swing arm scanning system/C-arm

102 patient table

103 robotic arm

104 X-ray tube

105 X-ray detector

106 X-ray

107 object of interest/patient

150 workstation/personal computer

155 control unit

160 data processing device

204 radiation source

205 radiation detector

206 radiation beam

207 object of interest/patient

210a translation direction

210b rotation direction

211 first image

212 second image

220 stitched image/composed image

220a stitched image/composed image (first choice)

220b stitched image/composed image (second choice)

220c stitched image/composed image (third choice)

235 overlap

a, b, c, d, e, f voxels of patient

304 radiation source

305 radiation detector

306 radiation beam

311 first image

312 second image

331 resampled image

332 resampled image

335 overlap

460 data processing device

461 central processing unit/image processor

462 memory

463 display device

464 keyboard

1. A method for collecting images of an object (107, 207) of interest for the purpose of image stitching in order to provide for an enlarged image field of view, the method comprising
acquiring a first image (211) of the object (107, 207) using first radiation (106, 206) being emitted from a radiation source (104, 204), being transmitted through the object (107, 207) and being detected by a radiation detector (105, 205), whereby the object (107, 207) is positioned relative to the radiation source (104, 204) in a first spatial position,
rotating the radiation detector (105, 205) around the radiation source (104, 204), and
acquiring a second image (212) of the object (107, 207) using second radiation (106, 206) being emitted from the radiation source (104, 204), being transmitted through the object (107, 207) and being detected by the radiation detector (105, 205), whereby
the object (107, 207) is positioned relative to the radiation source (104, 204) in a second spatial position, which is the same as the first spatial position.

2. A method according to claim 1, wherein
rotating the radiation detector (105, 205) is carried out in a circular manner.

3. A method according to claim 1, wherein
rotating the radiation detector (105, 205) comprises
maintaining the spatial position of the object (107, 207) relative to the radiation source (104, 204).

4. A method according to claim 1, wherein
rotating the radiation detector (105, 205) comprises
rotating both the radiation detector (105, 205) and the radiation source (104, 204) around a rotational axis, and
translating the object (107, 207) relative to the rotational axis such that the second spatial position of the radiation source is the same as the first spatial position of the radiation source.

5. The method according to claim 1, further comprising
joining the first image (211) and the second image (212) at a region of overlap (235) to form a stitched image having an image field of view larger than the field of view of the first image (211) or second image (212) individually.

6. The method according to claim 5, wherein
joining the first image (211) and the second image (212) comprises
determining the relative position between the first image (211) and the second image (212) by using a common geometry being identifiable within both the first image (211) and the second image (212).

7. The method according to claim 1, further comprising
resampling data representing the first image (311) and/or resampling data representing the second image (312) in order to simulate a planar common virtual detector plane (331, 332) for acquiring a first resampled image (311) and for acquiring a second resampled image (312).

8. The method according to claim 1, wherein
the first radiation (106, 206) and/or the second radiation (106, 206) is X-radiation.

9. A data processing device (460)
for collecting images of an object (107, 207) of interest for the purpose of image stitching in order to provide for an enlarged image field of view,
the data processing device (460) comprising
a data processor (461), which is adapted for performing the method as set forth in claim 1, and
a memory (462) for storing image data representing the first image (211) and/or the second image (212).

10. A medical system, in particular a C-arm system,
for collecting images of an object (107, 207) of interest for the purpose of image stitching in order to provide for an enlarged image field of view,
the medical system comprising
a data processing device (460) as set forth in claim 9.

11. A computer-readable medium on which there is stored a computer program
for collecting images of an object (107, 207) of interest for the purpose of image stitching in order to provide for an enlarged image field of view,
the computer program, when being executed by a data processor (461), is adapted for performing the method as set forth in claim 1.

12. A program element
for collecting images of an object (107, 207) of interest for the purpose of image stitching in order to provide for an enlarged image field of view,
the program element, when being executed by a data processor (461), is adapted for performing the method as set forth in claim 1.
